(19) United States
(12) Patent Application Publication
(10) Pub. No.: US 2008/0188858 A1
(43) Pub. Date: Aug. 7, 2008

(54) BONE TREATMENT SYSTEMS AND METHODS

(76) Inventors: Robert Luzzi, Pleasanton, CA (US); John H. Shadduck, Tiburon, CA (US); Csaba Truckai, Saratoga, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/024,969

(22) Filed: Feb. 1, 2008

Related U.S. Application Data
(60) Provisional application No. 60/899,487, filed on Feb. 5, 2007.

(51) Int. Cl. A61B 17/58

(52) U.S. Cl. 606/94; 606/92; 606/93

(57) ABSTRACT

Bone cement formulations are provided that have an extended working time for use in vertebroplasty procedures and other osteoplasty procedures. In one embodiment, a settable bone cement includes a polymerizable composition with a powder component comprising an X-Ray contrast medium and a liquid component, wherein the setting time of the cement is at least about 25 minutes, at least about 30 minutes, at least about 35 minutes, and at least about 40 minutes.

### CROSS-REFERENCE TO RELATED APPLICATIONS

This application claims the benefit of priority under 35 U.S.C. §119(e) of U.S. Provisional Application No. 60/899,487, filed on Feb. 5, 2007, entitled Bone Treatment Systems and Methods.

### BACKGROUND OF THE INVENTION

1. Field of the Invention

Embodiments of the present invention relate to bone cement formulations that have an extended working time for use in vertebroplasty procedures and other osteoplasty procedures. Further embodiments of the present invention relate to bone cement formulations having extended working time together with cement injectors that include energy delivery systems for on-demand control of cement flow viscosity and flow parameters.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80+ year-old women. Medical advances aimed at slowing or arresting bone loss from aging have not provided solutions to this problem. Further, the population affected grows steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts, and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of polymethyl methacrylate (PMMA) into a fractured vertebral body via a trocar and cannula. The targeted vertebrae are identified under fluoroscopy. A needle is introduced into the vertebrae body under fluoroscopic control to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique—and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, as compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture, and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82.

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

From the forgoing, then, there is a need to provide bone cements and methods for use in treatment of vertebral compression fractures that provide a greater degree of control over introduction of cement and that provide better outcomes.

### SUMMARY OF THE INVENTION

In an embodiment, a settable bone cement is provided. The bone cement comprises a polymerizable composition having a setting time of about 25 minutes or more. The bone cement further comprises a powder component comprising an X-Ray contrast medium and a liquid component.

In another embodiment, a bone cement is provided. The bone cement comprises a powder component and a liquid component. The powder component comprises about 64 to 75 wt. % PMMA, about 27 to 32 wt. % of an X-ray contrast medium, and about 0.4 to 0.8 wt. % benzoyl peroxide (BPO), where the amount of each is on the basis of the total weight of the powder component. The liquid component comprises greater than about 99 wt. % methyl methacrylate (MMA), less than about 1 wt. % N,N-dimethyl-p-toluidine (DMPT), and about 30 to 120 ppm hydroquinone, where the amount of each is on the basis of the total amount of the liquid component.

In an additional embodiment, a method for injecting a bone cement to treat an abnormality in a bone is provided. The method comprises providing a bone cement having a setting time of about 25 minutes or more, injecting the bone cement into a vertebra of a patient using a bone cement injector system, and applying energy to the bone cement from an energy emitter in the injector system to thereby increase the viscosity of the bone cement and accelerate the setting time of the bone cement.

In another embodiment, a method for anchoring an implant member in a bone with a bone cement is provided. The method comprises positioning an implant member in a bone of a patient. The method additionally comprises injecting a bone cement within the region of the bone-implant interface using an injector system. The bone cement has a setting time of about 25 minutes or more. The method further comprises applying energy to the bone cement from the injector system to thereby increase the viscosity of the bone cement and accelerate the setting time of the bone cement. In certain embodiments, the implant member comprises at least one of anchors, screws, plates, supports, ports, rods and a prosthesis.

In a further embodiment, a bone cement kit is provided. The bone cement kit comprises a bone cement and a bone cement injector. The bone cement comprises a powder component and a liquid component, where the powder component comprises polymethyl methacrylate (PMMA). The bone cement also provides a setting time of about 25 minutes or more. The bone cement injector is configured to apply energy to the bone cement to thereby increase the viscosity of the bone cement and accelerate said setting time of the bone cement.

### BRIEF DESCRIPTION OF THE DRAWINGS

In order to better understand the invention and to see how it may be carried out in practice, some embodiments are next described, by way of non-limiting examples only, with reference to the accompanying drawings, in which like reference characters denote corresponding features consistently throughout similar embodiments in the attached drawings.

FIG. 1 is a schematic view of a hydraulic bone cement injection system and sensing system in accordance with an embodiment of the invention.

FIG. 2 is another schematic view of the bone cement injector of FIG. 1.

FIG. 3A is a schematic sectional view of a vertebra showing a first step in an embodiment of method of the present disclosure.

FIG. 3B is a schematic sectional view of the vertebra of FIG. 3A showing a subsequent step in a method of the present disclosure.

FIG. 3C is a schematic sectional view similar to FIGS. 3A-3B showing a subsequent step in a method of the present disclosure wherein a sensing system detects a retrograde flow.

FIG. 4 is a, schematic view of another embodiment of a bone cement injector of the present disclosure.

FIG. 5 is a schematic, sectional view of a distal portion of the bone cement injector of FIGS. 1-2 with a thermal energy emitter in an interior bore of the injector, a sensor system, and scratch-resistant insulative exterior coating.

FIG. 6 is a plot illustrating setting time as a function of the concentration of BPO and DMPT present within embodiments of a bone cement composition.

FIG. 7 is a plot illustrating the temperature-time behavior of embodiments of the bone cement composition under conditions where the composition is and is not heated.

FIG. 8 is a plot illustrating the viscosity-time behavior of embodiments of the bone cement composition heated to temperatures ranging between about 25° C. to 55° C.

### DETAILED DESCRIPTION OF THE PREFERRED EMBODIMENTS

This application is related to the following U.S. patent and Provisional patent Applications: application Ser. No. 11/165,651 filed Jun. 24, 2005 titled Bone Treatment Systems and Methods; application Ser. No. 11/165,652 filed Jun. 24, 2005 titled Bone Treatment Systems and Methods; application Ser. No. 11/208,448 filed Aug. 20, 2005 titled Bone Treatment Systems and Methods; Application No. 60/713,521 filed Sep. 1, 2005 titled Bone Treatment Systems and Methods; and application Ser. No. 11/209,035 filed Aug. 22, 2005, titled Bone Treatment Systems and Methods. The entire contents of all of the above applications are hereby incorporated by reference and should be considered a part of this specification.

“Bone fill, fill material, or infill material or composition” includes its ordinary meaning and is defined as any material for infilling a bone that includes an in-situ hardenable material or that can be infused with a hardenable material. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is substantially unable to withstand a static shear stress and responds with an irrecoverable flow (a fluid)—unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Substantially” or “substantial” mean largely but not entirely. For example, substantially may mean about 10% to about 99.999%, about 25% to about 99.999% or about 50% to about 99.999%.

“Osteoplasty” includes its ordinary meaning and means any procedure wherein fill material is delivered into the interior of a bone.

“Vertebroplasty” includes its ordinary meaning and means any procedure wherein fill material is delivered into the interior of a vertebra.

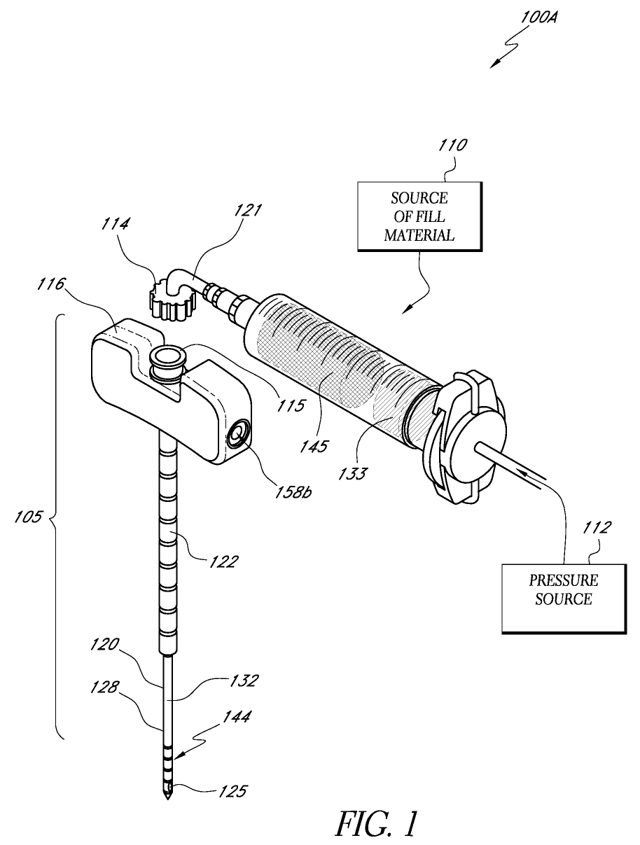

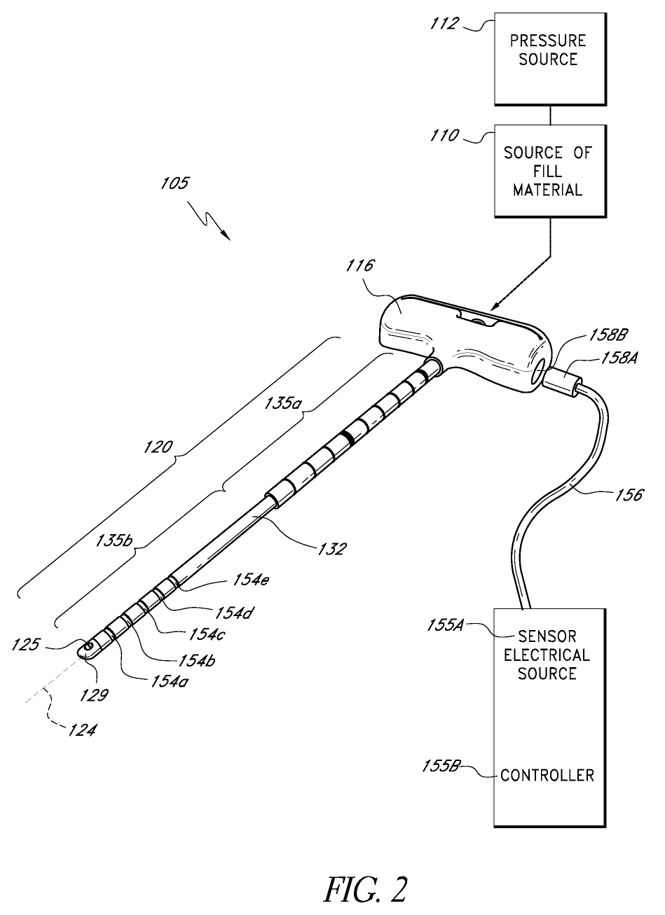

Referring to FIGS. 1-2, an embodiment of bone fill introducer or injector system 100A is shown that is configured for treatment of the spine in a vertebroplasty procedure. The system 100A includes a bone cement injector 105 that is coupled to a source 110 of a bone fill material wherein the injection of the fill material is carried out by a pressure mechanism or source 112 operatively coupled to source 110 of the bone fill material. The pressure source 112 can have a hydraulic actuator that is manually or computer controlled. For example, the fill material source 110 and pressure mechanism 112 can include a syringe, where the fill source 110 includes the barrel of the syringe and the pressure mechanism 112 includes the plunger of the syringe. The source 110 of fill material further includes a coupling or fitting 114 for sealable locking to a cooperating fitting 115 at a proximal end or handle 116 of the bone cement injector 105 that has an elongated introducer sleeve 120. In one embodiment, a syringe-type source 110 is coupled directly to fitting 115 with a flexible, rigid or bendable (deformable) hydraulic tube 121 extending to pressure source 112. The fill material then can flow through handle 116 to communicate with a passageway 122 in introducer sleeve 120.

In an embodiment, a vertebroplasty procedure using the system 100A would insert the injector 105 of the introducer system 100A of FIG. 1 through a pedicle of a vertebra for accessing the osteoporotic cancellous bone. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician uses a scalpel to make a 1 to 5 mm skin incision over each targeted pedicle. Thereafter, the injector 105 is advanced through the pedicle into the anterior region of the vertebral body, which typically is the region of greatest compression and fracture. The physician confirms the introducer path posterior to the pedicle, through the pedicle and within the vertebral body, by anteroposterior and lateral X-Ray projection fluoroscopic views. The introduction of infill material as described below can be imaged several times, or continuously, during the treatment depending on the imaging method.

In FIGS. 1-5, it can be seen that elongated introducer sleeve 120 of bone cement injector 105 includes interior passageway 122 extending about axis 124 wherein the passageway 122 terminates in a distal open outlet 125. The outlet 125 can be a single opening or a plurality of openings about the radially outward surface 128 of sleeve 120 or an opening at the distal tip 129 of the sleeve. The distal tip 129 can be blunt or sharp. In one embodiment, a core portion 130 of sleeve 120 is an electrically conductive metal sleeve, such as a stainless steel hypo tube. The core sleeve portion 130 has both an exterior insulative coating 132 and an interior insulative coating 232 that will be described in greater detail below.

In one embodiment, as shown in FIGS. 1-2, the bone fill system 100A has a container of fill material source 110 that is pressurized by a hydraulic source acting on a floating piston 133 (phantom view) in the source 110. In FIGS. 1-2, it can be seen that introducer sleeve 120 has a proximal portion 135a that is larger in cross-section than distal portion 135b with corresponding larger and smaller interior channel portions therein. This allows for lesser injection pressures since the cement flow needs to travel less distance through the smallest diameter distal portion of the introducer sleeve 120. The distal portion 135b of the introducer sleeve 120 can have a cross section ranging between about 2 mm and 4 mm with a length ranging between about 40 mm and 60 mm. The proximal portion 135a of introducer sleeve 120 can have a cross-section ranging between about 5 mm and 15 mm, or between about 6 mm and 12 mm.

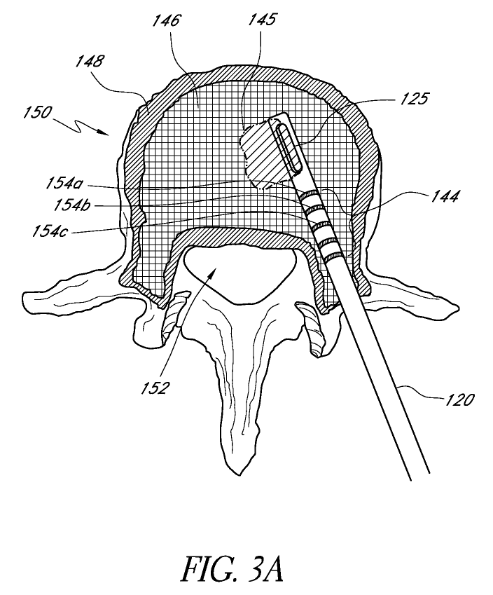

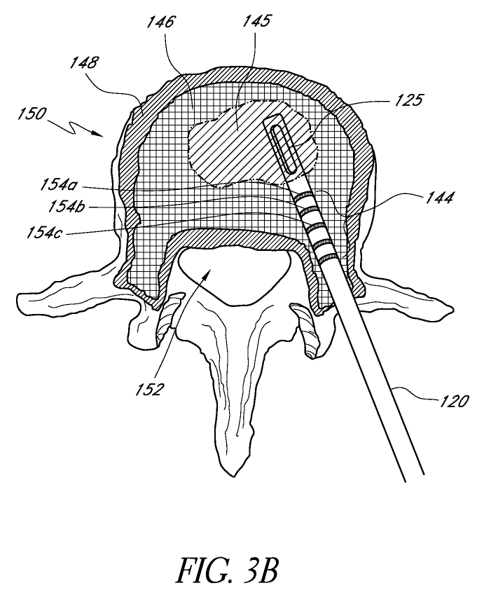

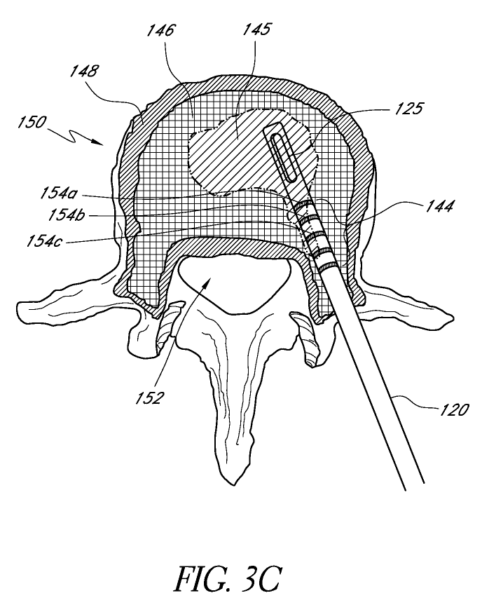

As can be seen in FIGS. 1-2, the exterior surface of introducer sleeve 120 carries a sensor system 144 that is adapted to sense the flow or movement of a fill material or cement 145 (see FIGS. 3A-3C) proximate to the sensor system 144. The introducer sleeve 120 with such a sensor system 144 is particularly useful in monitoring and preventing extravasation of fill material 145 in a vertebroplasty procedure.

In one embodiment and method of use, referring to FIGS. 3A-3C, the introducer sleeve 120 is used in a conventional vertebroplasty with a single pedicular access or a bi-pedicular access. The fill material 145 is a bone cement, such as PMMA, that is injected into cancellous bone 146 which is within the interior of the cortical bone surface 148 of vertebra 150.

In FIGS. 3A-3B, it can be seen that a progressive flow of cement 145 is provided from outlet 125 of introducer sleeve 120 into the interior of the vertebra 150. FIG. 3A illustrates an initial flow volume, with FIG. 3B illustrating an increased flow volume of cement 145. FIG. 3C depicts a situation that is known to occur wherein bone is fractured along the entry path of introducer 120 wherein the cement 145 under high injection pressures finds the path of least resistance to be at least partly in a retrograde direction along the surface of introducer sleeve 120. The retrograde flow of cement as in FIG. 3C, if allowed to continue, could lead to cement extravasation into the spinal canal 152 which can lead to serious complications. As can be understood from FIG. 3C, the sensor system 144 is configured to be actuated when cement 145 comes into contact with the sensor system 144.

In one embodiment, as shown in FIGS. 2-3C, the sensor system 144 comprises a plurality of spaced apart exposed electrodes or electrode portions (e.g., electrodes 154a, 154b, 154c, 154e) coupled to sensor electrical source 155A via cable or lead 156 and plug 158a. The plug 158a is connected to electrical connector 158b in the proximal handle end of the introducer wherein the electrical source carries a low voltage direct current or Rf current between the opposing potentials of spaced apart electrodes. The voltage can be from about 0.1 volt to 500 volts, or from about 1 volt to 5 volts, and creates a current path through the tissue between a pair of electrodes. The current can be continuous, intermittent and/or multiplexed between different electrode pairs or groups of electrodes.

The configuration of the electrodes can be varied, as necessary. For example, the arrangement of electrodes can be axially spaced apart ring-type electrodes as shown in FIGS. 1 and 2, or the electrodes can be discrete elements, helically spaced electrodes. The electrodes can further be miniaturized electrodes, as in thermocouples, MEMS devices, or any combination thereof. The number of sensors 144 or electrodes can range from about 1 to 100 and can be adapted to cooperate with a ground pad or other surface portion of sleeve 120. In one embodiment, the electrodes can include a PTC or NTC material (positive temperature coefficient of resistance or negative temperature coefficient of resistance) to thereby function as a thermistor to allow measurement of temperature as well as functioning as a sensor. The sensor system 144 includes a controller 155B (FIG. 2) that measures at least one selected parameter of the current flow to determine a change in a parameter (e.g., impedance). When the non-conductive bone cement 145 contacts one or more electrodes of the sensor system 144, the controller 155B identifies a change in the selected electrical parameter and generates a signal to the operator. The scope of the invention includes sensor systems capable of sensing a change in electrical properties, reflectance, fluorescence, magnetic properties, chemical properties, mechanical properties or a combination thereof.

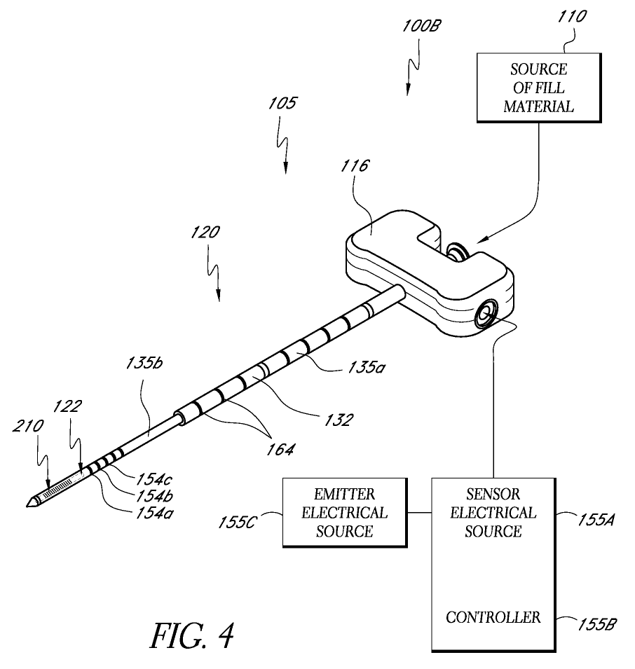

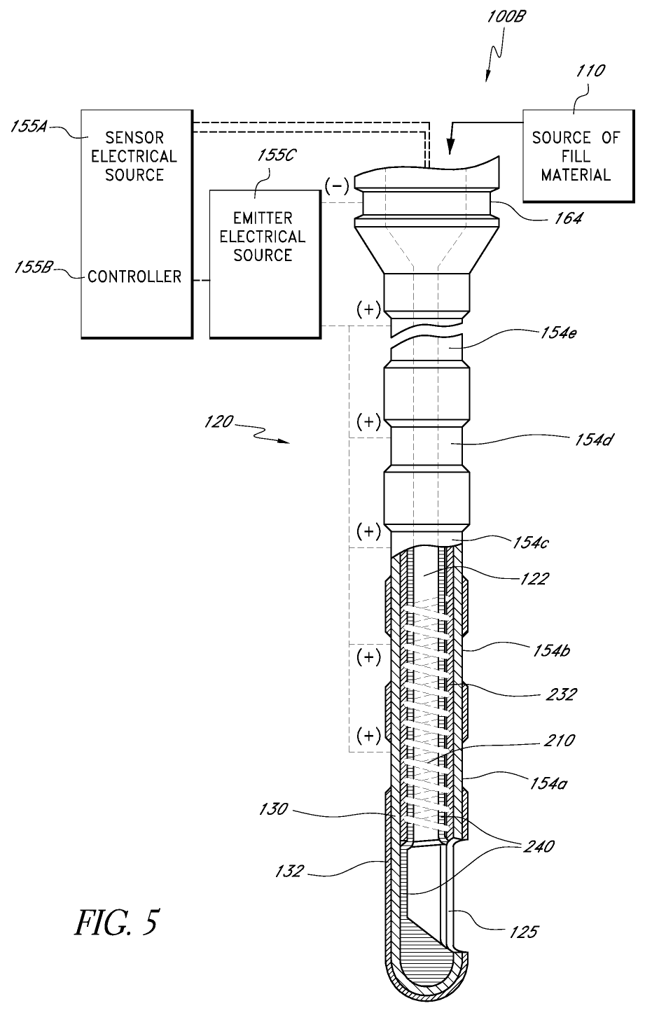

Now referring to FIGS. 4 and 5, an alternative system 100B includes a bone cement injector 105 that is similar to the injector of FIGS. 1-2, but with a different embodiment of sensor system together with an additional energy delivery system for applying energy to fill material for altering its viscosity. In this embodiment, the ring electrode portions (i.e. electrodes 154a, 154b, 154c, 154d, 154e in phantom view) are exposed portions of the metal core portion 130 of the sleeve 120 (see FIG. 5) that is coupled via a lead 156 to electrical source 155A. The electrode portions 154a, 154b, 154c, 154d, 154e are indicated having a first polarity (+) that cooperate with one or more second polarity (−) return electrodes 164 in a more proximal portion of the sleeve coupled by lead 156 to the sensor electrical source 155A. In this embodiment, current flows through the multiple electrode portions 154a, 154b, 154c, 154d, 154e and then though engaged tissue to the return electrodes 164, wherein the current flow signals certain impedance parameters before and during an initial injection of cement 145, as in FIGS. 3A-3B. When there is a retrograde flow of cement 145, as in FIG. 3C, that covers one or more electrode portions 154a, 154b, 154c, 154d, 154c then the electrical parameter (e.g., impedance) changes to thus signal the operator that such a retrograde flow has contacted or covered an electrode portion 154a, 154b, 154c, 154d, 154e. The change in parameter can be a rate of change in impedance, a change in impedance compared to a data library, etc. which signals the operator of such a flow and wherein controller 155B also can automatically terminate the activation of pressure source 112.

In the system of FIGS. 4 and 5, the bone fill injection system 100B further includes a thermal energy emitter within a distal portion of interior passageway 122 of the introducer 120 for heating a flow of bone cement from an open termination 125 in the introducer. In one embodiment, the thermal energy emitter is a resistive heating element 210 configured to elevate the temperature of cement 145 to at least 50° C., at least 60° C., at least 70° C., or at least 80° C. The resistive element 210 is coupled to emitter electrical source 155C as depicted in FIGS. 4 and 5, together with a controller 155B. The controller 155B is further configured to control cement inflow parameters such as variable flow rates, constant flow rates and/or pulsed flows in combination with controlled energy delivery. The thermal energy delivery is adapted to accelerate polymerization and increase the viscosity of a PMMA or similar bone cement as disclosed in the co-pending U.S. patent Applications listed below.

In another embodiment, the thermal energy emitter also can be an Rf emitter. The Rf emitter is adapted for ohmically heating a bone cement that carries electrically conductive compositions as disclosed in the below co-pending U.S. patent application Ser. No. 11/165,652 filed Jun. 24, 2005; Ser. No. 11/165,651 filed Jun. 24, 2005; Ser. No. 11/208,448 filed Aug. 20, 2005; and Ser. No. 11/209,035 filed Aug. 22, 2005, the entirety of which are hereby incorporated by reference in their entirety.

In another embodiment, the thermal energy emitter can be configured for delivering thermal energy to bone cement. The thermal energy emitter may comprise a resistively heated emitter, a light energy emitter, an inductive heating emitter, an ultrasound source, a microwave emitter, any electromagnetic energy emitter to cooperate with the bone cement, and combinations thereof.

In the embodiments of FIGS. 4 and 5, the controller 155B is adapted to control all parameters of (i) heating the bone cement, (ii) the cement injection pressure and/or flow rate, (iii) energy delivery to cement flows in or proximate the distal end of the introducer and (iv) energy delivery to sense retrograde flows about the exterior surface of the introducer.

In one embodiment, depicted in FIG. 5, the resistive heating element 210 comprises a helically wound coil of a resistive material positioned within the interior passageway 122 of the introducer 120. As can be seen in FIG. 5, the heating element 210 can be carried within an insulative coating 232 within the interior of core sleeve 130 which is a conductive metal as described above.

In another embodiment, still referring to FIG. 5, the heating element 210 is formed of a polymer positive temperature coefficient of resistance (PTCR) material and coupled to a suitable voltage source to provide a constant temperature heater as is known in the art. The electrical leads from the voltage source can be coupled to opposing ends of the PTCR heating element 210, or opposing sides of the heating element, or any spaced apart portions of the heating element 210. In one embodiment, the electrical leads have terminal portions that extend substantially over surface portions of the PTCR heating element as when the terminal portions are a conductive ink or similar adherent conductive material.

FIG. 5 illustrates another embodiment, where it can be seen that the exterior surface of sleeve 120 has an insulative, scratch-resistant coating indicated at 132 that comprises a thin layer of an insulative amorphous diamond-like carbon (DLC) or a diamond-like nanocomposite (DCN). It has been found that such coatings have high scratch resistance, as well as lubricious and non-stick characteristics that are useful in bone cement injectors of the present disclosure. Such coatings are particularly useful for an introducer sleeve 120 configured for carrying electrical current for (i) impedance sensing purposes; (ii) energy delivery to bone fill material; and/or (iii) ohmic heating of tissue. For example, when inserting a bone cement injector through the cortical bone surface of a pedicle and then into the interior of a vertebra, it is important that the exterior insulative coating portions do not fracture, chip or scratch to thereby insure that the electrical current carrying functions of the injector are not compromised.

Embodiments of the amorphous diamond-like carbon coatings and the diamond-like nanocomposites are available from Bekaert Progressive Composites Corporations, 2455 Ash Street, Vista, Calif. 92081 or its parent company or affiliates. Further information on the coating can be found at the website of Beckaert Group by selecting diamond-like films under the products section. The diamond-like coatings comprise amorphous carbon-based coatings with high hardness and low coefficient of friction. The amorphous carbon coatings exhibit non-stick characteristics and excellent wear resistance. The coatings are thin, chemically inert, and have a very low surface roughness. In one embodiment, the coatings have a thickness ranging between about 0.001 mm and 0.010 mm; or between about 0.002 mm and 0.005 mm. The diamond-like carbon coatings are a composite of sp2 and sp3 bonded carbon atoms with a hydrogen concentration between about 0 and 80%. Another diamond-like nanocomposite coating (a-C:H/a-Si:O; DLN) is made by Bakaert and is suitable for use in the bone cement injector of the embodiments disclosed herein. The materials and coatings are known by the names Dylyn® Plus, Dylyr®/DLC, and Cavidur®.

FIG. 5 further illustrates another aspect of bone cement injector 105 that again relates to the thermal energy emitter (resistive heater 210) within interior passageway 122 of introducer 120. In one embodiment, it has been found that it is advantageous to provide a lubricious surface layer 240 within the interior of resistive heater 210 to insure uninterrupted cements flows through the thermal emitter without sticking. In one embodiment, surface layer 240 is a fluorinated polymer such as Teflon® or polytetrafluroethylene (PTFE). Other suitable fluoropolymer resins can be used such PFA (Perfluoroalkoxy copolymer), FEP (Fluorinated ethylenepropylene), ECTFE (Ethylenechlorotrifluoroethylene), ETFE, Polyethylene, Polyamide, PVDF, Polyvinyl chloride, and silicone. The scope of the present disclosure includes providing a bone cement injector having a flow channel extending therethrough with at least one open termination 125, where a surface layer 240 within the flow channel has a static coefficient of friction of less than about 0.5, less than about 0.2, or less than about 0.1.

In another embodiment, the bone cement injector has a passageway 122 extending therethrough with at least one open termination 125, where at least a portion of the surface layer 240 of the passageway is ultra-hydrophobic or hydrophobic, which may better prevent a hydrophilic cement from sticking.

In another embodiment, the bone cement injector has a passageway 122 extending therethrough with at least one open termination 125, wherein at least a portion of the surface layer 240 of the flow channel is hydrophilic, which may prevent a hydrophobic cement from sticking.

In another embodiment, the bone cement injector has a passageway 122 extending there through with at least one open termination 125 in a distal end thereof, wherein the surface layer 240 of the flow channel has at least one of a high dielectric strength, a low dissipation factor, and a high surface resistivity.

In another embodiment, the bone cement injector has a passageway 122 extending there through with at least one open termination 125 in a distal end thereof, wherein the surface layer 240 of the flow channel is oleophobic. In another embodiment, the bone cement injector has a passageway 122 extending there through with at least one open termination 125 in a distal end thereof, wherein the surface layer 240 of the flow channel has a substantially low coefficient of friction polymer or ceramic.

In another embodiment, the bone cement injector has a passageway 122 extending there through with at least one open termination 125 in a distal end thereof, wherein the surface layer 240 of the flow channel has a wetting contact angle greater than about 70°, greater than about 85°, and greater than about 100°.

In another embodiment, the bone cement injector has a passageway 122 extending there through with at least one open termination in a distal end thereof, wherein the surface layer 240 of the flow channel has an adhesive energy of less than about 100 dynes/cm, less than about 75 dynes/cm, and less than about 50 dynes/cm.

The apparatus above also can be configured with any other form of thermal energy emitter that includes the non-stick and/or lubricious surface layer as described above. In one embodiment, the thermal energy emitter can comprise at least in part an electrically conductive polymeric layer. In one such embodiment, the electrically conductive polymeric layer has a positive temperature coefficient of resistance.

Further embodiments of the present disclosure relate to bone cement compositions and formulations for use in the bone cement delivery systems described above. The bone cement formulations provide for an extended working time, since the viscosity can be altered and increased on demand when injected.

Bone cements, such as polymethyl methacrylate (PMMA), have been used in orthopedic procedures for several decades, principally for anchoring endoprostheses in a bone. For example, skeletal joints such as in the hip are replaced with a prosthetic joint. About one million joint replacement operations are performed each year in the U.S. Frequently, the prosthetic joint is cemented into the bone using an acrylic bone cement such as PMMA. In recent years, bone cements also have been widely used in vertebroplasty procedures wherein the cement is injected into a fractured vertebra to stabilize the fracture and eliminate micromotion that causes pain.

Polymethyl methacrylate bone cement, prior to injection, comprises a powder component and a liquid monomer component. The powder component comprises granules of methyl methacrylate or polymethyl methacrylate, an X-ray contrast agent and a radical initiator. Typically, barium sulfate or zirconium dioxide is used as an X-ray contrast agent. Benzoyl peroxide (BPO) is typically used as radical initiator. The liquid monomer component typically consists of liquid methyl methacrylate (MMI), an activator, such as N,N-dimethyl-p-toluidine (DMPT) and a stabilizer, such as hydroquinone (HQ). Just prior to injecting PMMA bone cements, the powder component and the monomer component are mixed and thereafter the bone cement hardens within several minutes following radical polymerization of the monomer.

Typical bone cements formulations (including PMMA formulations) used for vertebroplasty have a fairly rapid cement curing time after mixing of the powder and liquid components. This allows the physician to not waste time waiting for the cement to increase in viscosity prior to injection. Further, the higher viscosity cement is less prone to unwanted extravasation which can cause serious complications. The disadvantage of such current formulations is that the “working time” of the cement is relatively short—for example about 5 to 8 minutes—in which the cement is within a selected viscosity range that allows for reasonably low injection pressures while still being fairly viscous to help limit cement extravasation. In one embodiment, the viscosity ranges between approximately 50 to 500 N s/m² and is measured according to ASTM standard F451, “Standard Specification for Acrylic Bone Cement,” which is hereby incorporated by reference in its entirety.

In one embodiment, the bone cement of the present disclosure provides a formulation adapted for use with the cement injectors and energy delivery systems described above. These formulations are distinct from conventional formulations and have greatly extended working times for use in vertebroplasty procedures with the “on-demand” viscosity control methods and apparatus disclosed herein and in co-pending applications listed and incorporated by reference above.

In one embodiment, the bone cement provides a formulation adapted for injection into a patient's body, wherein the setting time is about 25 minutes or more, more preferably about 30 minutes or more, more preferably about 35 minutes or more, and even more preferably about 40 minutes or more. Setting time is measured in accordance with ASTM standard F451.

In one embodiment, the bone cement of the present disclosure, prior to mixing and setting, comprises a powder component and a liquid component. The powder component comprises a PMMA that is about 64% to 75% by weight based on overall weight of the powder component. In this formulation, an X-ray contrast medium is about 27% to 32% by weight based on overall weight of the powder component. The X-ray contrast medium, in one embodiment, comprises barium sulfate (BaSO₄) or zirconium dioxide (ZrO₂). This formulation further includes BPO that is about 0.4% to 0.8% by weight based on overall weight of the powder component. In this formulation, the liquid component includes MMA that is greater than about 99% by weight based on overall weight of the liquid component. In this formulation, the liquid component includes DMPT that is less than about 1% by weight based on overall weight of the liquid component. In this formulation, the liquid component includes hydroquinone that ranges between about 30 and 120 ppm of the liquid component. In this formulation, the liquid weight/powder weight ratio is equal to or greater than about 0.4. In this formulation, the PMMA comprises particles having a mean diameter ranging from about 25 microns to 200 microns or ranging from about 50 microns to 100 microns.

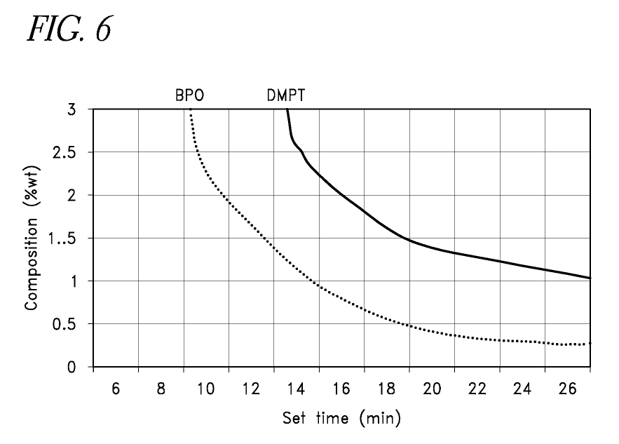

In certain embodiments, the concentrations of benzoyl peroxide and DMPT may be varied in order to adjust setting times. Studies examining the influence of bone cement concentration on setting times (FIG. 6) have demonstrated that, in bone cements comprising BPO and DMPT, increases in BPO and DMPT concentration increase the set time of the bone cement. The data further illustrate that, of the two bone cement constituents, BPO has a greater rate of effect on set time than does DMPT. Thus, in certain embodiments of the bone cement composition, the concentration of BPO, DMPT, and combinations thereof, may be increased within the ranges discussed above so as to increase the setting time of the composition.

The setting time of the cement may also be influenced by applying energy to the bone cement composition. As discussed above, embodiments of the injector 105 may be configured to deliver energy to the bone cement composition. In certain embodiments, the applied energy may heat the bone cement composition to a selected temperature.

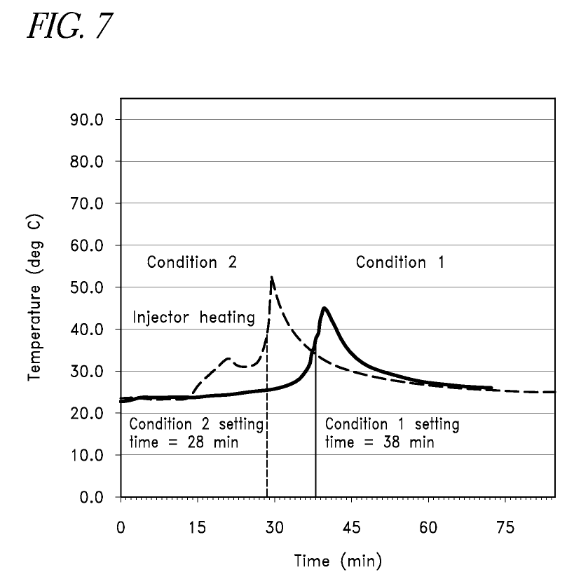

FIG. 7 illustrates the temperature as a function of time from initial mixing for one embodiment of the bone composition so injected. The solid line of FIG. 7 represents the behavior of the composition when it is not heated by the injector 105, referred to as condition 1. It is observed that, under condition 1, the composition exhibits three regimes. The first regime is low heating rate regime, where the temperature of the composition increases modestly with time. In this regime, the composition begins to slowly self-heat due the onset of a chemical reaction between at least a portion of its components. The second regime is a high heating rate regime, where the chemical reaction causes the composition temperature rises sharply. Once the temperature of the composition peaks, the composition enters a third, cooling regime, during which the temperature of the composition decreases back to room temperature.

The dotted line of FIG. 7 represents the behavior of the composition when it is heated by the injector 105, referred to as condition 2. In contrast to condition 1, four regimes of behavior are exhibited by the composition under condition 2. The first, low heating rate regime, the second, high heating rate regime, and the third, cooling regime, are again observed. In contrast with condition 1, however, a new, injector heating regime, is observed between the first and second regimes. This new regime exhibits a rapid increase in the composition temperature due to injector heating of the composition. Although the composition temperature is observed to peak and fall towards the end of the duration of this regime, the temperature does not fall back to the same level as observed under condition 1 at about the same time. Therefore, when the second, high heating rate regime is entered, the temperature of the composition under condition 2 is greater than that under condition 1 and the composition temperature rises to a peak temperature which is greater than that achieved under condition 1.

The setting time of the compositions under conditions 1 and 2 can be measured according to ASTM standard F451 and compared to identify changes in setting time between the two conditions. It is observed that the setting time of the composition under condition 1 is approximately 38 minutes, while the setting time of the composition under condition 2 is approximately 28 minutes, a reduction of about 10 minutes. Thus, by heating the bone cement, the setting time of embodiments of the bone cement composition may be reduced.

From the forgoing, then, it can be appreciated that by varying the BPO and/or DMPT concentrations of the bone cement composition or by heating the bone cement composition, the setting time of the bone cement may be increased or decreased. Furthermore, in certain embodiments, the concentration of BPO and/or DMPT in the bone cement may be varied and the composition may be heated so as to adjust the setting time to a selected value. As discussed above, in certain embodiments, the setting time is selected to be about 25 minutes or more, more preferably about 30 minutes or more, more preferably about 35 minutes or more, and even more preferably about 40 minutes or more.

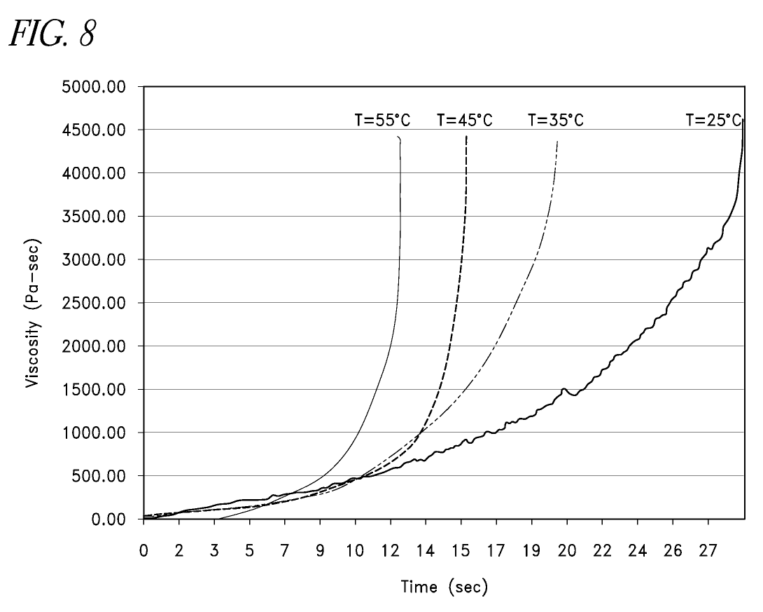

Embodiments of the bone cement composition may further be heated using the injector 105 in order to alter the viscosity of the composition. FIG. 8 illustrates measurements of viscosity as a function of time for an embodiment of the bone cement compositions heated to temperatures ranging between about 25° C. to 55° C. It may be observed that the bone cement at the lowest temperature, 25° C., exhibits the slowest rate of viscosity increase, while the bone cement at the highest temperature, 55° C., exhibits the highest rate of viscosity increase. Furthermore, at intermediate temperatures, the bone cement exhibits intermediate rates of viscosity increase.

From the behavior of condition 1 in FIG. 7, it can be seen that the peak temperature of the bone cement composition is higher when the cement is heated by the injector 105. Furthermore, by adjusting the energy output of the injector 105, the temperature to which the bone cement rises may be varied. Thus, embodiments of the injector 105 may be employed to deliver bone cements having selected levels of viscosity.

In another embodiment, the step of applying thermal energy as described above is accomplished by light energy from an LED, or from at least one of coherent light and non-coherent light. Such light energy source can have any suitable wavelength for applying or causing thermal effects in the bone cement flows. In one embodiment, a plurality of optic fibers can extend axially within wall of the cement injector sleeve with a distal fiber portion configured for propagation of light into the interior channel by cladding removal or other “side-firing” means known in the art.

In related methods, the system of the present disclosure can use any suitable energy source to accomplish the purpose of altering the viscosity of the fill material 145. The method of altering fill material can comprise at least one of a radiofrequency source, a laser source, a microwave source, a magnetic source and an ultrasound source. Each of these energy sources can be configured to preferentially deliver energy to a cooperating, energy sensitive filler component carried by the fill material. For example, such filler can be suitable chromophores for cooperating with a light source, ferromagnetic materials for cooperating with magnetic inductive heating means, or fluids that thermally respond to microwave energy.

The scope of the invention includes using additional filler materials such as porous scaffold elements and materials for allowing or accelerating bone ingrowth. In any embodiment, the filler material can comprise reticulated or porous elements of the types disclosed in co-pending U.S. patent application Ser. No. 11/146,891, filed Jun. 7, 2005, titled “Implants and Methods for Treating Bone” which is incorporated herein by reference in its entirety and should be considered a part of this specification. Such fillers also can carry bioactive agents. Additional fillers, or the conductive filler, also can include thermally insulative solid or hollow microspheres of a glass or other material for reducing heat transfer to bone from the exothermic reaction in a typical bone cement component.

The above description of certain embodiments is intended to be illustrative and not exhaustive. Particular characteristics, features, dimensions and the like that are presented in dependent claims can be combined and fall within the scope of the present disclosure. The disclosure also encompasses embodiments as if dependent claims were alternatively written in a multiple dependent claim format with reference to other independent claims. Specific characteristics and features of the disclosure and its method are described in relation to some figures and not in others, and this is for convenience only. While the principles of the present disclosure have been made clear in the exemplary descriptions and combinations, it will be obvious to those skilled in the art that modifications may be utilized in the practice of the present disclosure, and otherwise, which are particularly adapted to specific environments and operative requirements without departing from the principles of the disclosed embodiments. The appended claims are intended to cover and embrace any and all such modifications, with the limits only of the true purview, spirit and scope of the disclosure.

Of course, the foregoing description is that of certain features, aspects and advantages of the present disclosure, to which various changes and modifications can be made without departing from the spirit and scope of the present disclosure. Moreover, the bone treatment systems and methods need not feature all of the objects, advantages, features and aspects discussed above. Thus, for example, those skilled in the art will recognize that the disclosed embodiments can be embodied or carried out in a manner that achieves or optimizes one advantage or a group of advantages as taught herein without necessarily achieving other objects or advantages as may be taught or suggested herein. In addition, while a number of variations of the disclosed embodiments have been shown and described in detail, other modifications and methods of use, which are within the scope of this disclosure, will be readily apparent to those of skill in the art based upon this disclosure. It is contemplated that various combinations or subcombinations of these specific features and aspects of embodiments may be made and still fall within the scope of the disclosure. Accordingly, it should be understood that various features and aspects of the disclosed embodiments can be combined with or substituted for one another in order to form varying modes of the discussed bone treatment systems and methods.

What is claimed is:

1. A settable bone cement, comprising:
a polymerizable composition comprising:
a powder component comprising an X-Ray contrast medium and;
a liquid component;
wherein the cement has a setting time of about 25 minutes or more.

2. A bone cement according to claim 1, wherein the powder component further comprises polymethyl methacrylate (PMMA) that is about 64% to 75% by weight based on overall weight of the powder component.

3. A bone cement according to claim 1, wherein the amount of X-ray contrast medium is about 27% to 32% by weight based on overall weight of the powder component.

4. A bone cement according to claim 1, wherein the X-ray contrast medium is selected from the group consisting of barium sulfate (BaSO₄) and zirconium dioxide (ZrO₂).

5. A bone cement according to claim 1, further including benzoyl peroxide (BPO) that is about 0.4% to 0.8% by weight based on overall weight of the powder component.

6. A bone cement according to claim 1, wherein the liquid component comprises methyl methacrylate (MMA) that is greater than about 99% by weight based on overall weight of the liquid component.

7. A bone cement according to claim 1, wherein the liquid component comprises N,N-dimethyl-p-toluidine (DMPT) that is less than about 1% by weight based on overall weight of the liquid component.

8. A bone cement according to claim 1, wherein the liquid component includes hydroquinone that ranges between about 30 and 120 ppm of the liquid component.

9. A bone cement according to claim 1, wherein the liquid weight/powder weight ratio is equal to or greater than about 0.4.

10. A bone cement according to claim 1, wherein the PMMA comprises particles having a mean diameter ranging from about 25 microns to 200 microns.

11. A bone cement according to claim 1, wherein the PMMA comprises particles having a mean diameter ranging from about 50 microns to 100 microns.

12. A bone cement according to claim 1, further comprising a filler selected from at least one of chromophores, ferromagnetic materials, a fluid that thermally responds to microwaves, a bone ingrowth accelerator, a bioactive agent, and a thermally insulating solid.

13. A settable bone cement comprising:
a powder component comprising polymethyl methacrylate (PMMA) and;
a liquid component;
wherein the bone cement provides a setting time of at least 25 minutes.

14. A settable bone cement as in claim 13, wherein the bone cement provides a setting time of at least 30 minutes.

15. A settable bone cement as in claim 13, wherein the bone cement provides a setting time of at least 35 minutes.

16. A settable bone cement as in claim 13, wherein the bone cement provides a setting time of at least 40 minutes.

17. A bone cement comprising:
a powder component comprising:
about 64 to 75 wt. % PMMA;
about 27 to 32 wt. % of an X-ray contrast medium; and
about 0.4 to 0.8 wt. % benzoyl peroxide (BPO);
wherein the amount of each is on the basis of the total weight of the powder component; and
a liquid component comprising:
greater than about 99 wt. % methyl methacrylate (MMA);
less than about 1 wt. % N,N-dimethyl-p-toluidine (DMPT), about 30 to 120 ppm hydroquinone;
wherein the amount of each on the basis of the total amount of the liquid component.

18. The bone cement according to claim 17, wherein the ratio of the weight of the liquid component to the weight of the powder component is equal to or greater than about 0.4.

19. A bone cement according to claim 17, further comprising a filler selected from at least one of chromophores, ferromagnetic materials, a fluid that thermally responds to microwaves, a bone ingrowth accelerator, a bioactive agent, and a thermally insulating solid.

20. A method for injecting a bone cement to treat an abnormality in a bone, comprising:
providing a bone cement having a setting time of about 25 minutes or more;
injecting said bone cement into a vertebra of a patient using a bone cement injector system; and
applying energy to the bone cement from an energy emitter in the injector system to thereby increase the viscosity of the bone cement and accelerate said setting time.

21. The method as in claim 20, wherein the bone cement comprises:
a powder component comprising:
about 64 to 75 wt. % PMMA;
about 27 to 32 wt. % of an X-ray contrast medium; and
about 0.4 to 0.8 wt. % benzoyl peroxide (BPO);
wherein the amount of each is on the basis of the total weight of the powder component; and
a liquid component comprising:
greater than about 99 wt. % methyl methacrylate (MMA);
less than about 1 wt. % N,N-dimethyl-p-toluidine (DMPT), about 30 to 120 ppm hydroquinone;
wherein the amount of each is on the basis of the total amount of the liquid component.

22. The method of claim 20, wherein the energy emitter comprises a thermal energy emitter configured to deliver thermal energy to the bone cement.

23. The method of claim 22, wherein the thermal energy emitter comprises at least one of a resistive heating element, an Rf emitter, a light energy emitter, an inductive heating emitter, an ultrasound emitter, and a microwave emitter.

24. The method of claim 20, wherein the X-ray contrast medium is selected from the group consisting of barium sulfate (BaSO₄) and zirconium dioxide (ZrO₂).

25. The method of claim 20, wherein the liquid weight/powder weight ratio is equal to or greater than about 0.4.

26. The method of claim 20, further comprising providing a bone cement having a setting time of at least 30 minutes.

27. The method of claim 20, further comprising providing a bone cement having a setting time of at least 35 minutes.

28. The method of claim 20, further comprising providing a bone cement having a setting time of at least 40 minutes.

29. A method for anchoring an implant member in a bone with a bone cement, comprising:
positioning an implant member in a bone of a patient;
injecting a bone cement within the region of the bone-implant interface with an injector system, wherein said bone cement has a setting time of about 25 minutes or more; and
applying energy to the bone cement from the injector system to thereby increase the viscosity of the bone cement and accelerate said setting time.

30. The method as in claim 29, wherein the bone cement comprises:
a powder component comprising:
about 64 to 75 wt. % PMMA;
about 27 to 32 wt. % of an X-ray contrast medium; and
about 0.4 to 0.8 wt. % benzoyl peroxide (BPO);
wherein the amount of each is on the basis of the total weight of the powder component; and
a liquid component comprising:
greater than about 99 wt. % methyl methacrylate (MMA);
less than about 1 wt. % N,N-dimethyl-p-toluidine (DMPT), about 30 to 120 ppm hydroquinone;
wherein the amount of each on the basis of the total amount of the liquid component.

31. The method as in claim 29, wherein the implant member comprises at least one of anchors, screws, plates, supports, ports, rods and a prosthesis.

32. A bone cement kit comprising:
a bone cement, comprising a powder component, comprising polymethyl methacrylate (PMMA), and a liquid component, wherein the bone cement provides a setting time of about 25 minutes or more; and
a bone cement injector system configured to apply energy to the bone cement to thereby increase the viscosity of the bone cement and accelerate said setting time of said bone cement.

33. A bone cement kit according to claim 32, wherein the bone cement injector system includes a positive temperature coefficient of resistance (PTCR) material operatively coupled to a voltage source.

34. A bone cement kit according to claim 32, wherein the bone cement injector system is operatively coupled to at least one energy source comprising an electromagnetic energy source, a radiofrequency source, a resistive heating source, a coherent light source, a non-coherent light source, a microwave source, a magnetic source, and an ultrasound source.
